(19) United States
(12) Patent Application Publication
(10) Pub. No.: US 2014/0273141 A1
(43) Pub. Date: Sep. 18, 2014

(54) Integrated Biorefinery

(71) Applicant: Algae Systems, LLC, (US)

(72) Inventor: Matthew Atwood, Chicago, IL (US)

(21) Appl. No.: 13/829,543

(22) Filed: Mar. 14, 2013

(51) Int. Cl. C12P 5/02

(52) U.S. Cl. CPC C12P 5/02
USPC 435/167; 435/290.4

(57) ABSTRACT

Integrated biorefineries are provided that integrate biomass cultivation with processing to convert cultivated biomass to a fuel, and further integrate carbon recovery from the biomass processing. Biomass processing to fuels begins in a treatment system, such as a hydrothermal treatment system, that produces an organic phase that is suitable for refining to a fuel and also produces a waste stream. The biorefinery optionally includes a dewatering system, a refining system to convert the organic phase to the fuel, and a cogeneration system configured to use at least some of the fuel produced by the treatment system to generate electricity and heat that can be used for biorefinery operations.

### CROSS-REFERENCE TO RELATED APPLICATIONS

This application is related to U.S. patent application Ser. No. 13/828,143 filed on Mar. 14, 2013 and entitled “Dewatering Systems and Methods for Biomass Concentration” which claims the benefit of U.S. Provisional Patent Application No. 61/664,532 filed on Jun. 26, 2012 and entitled “Dewatering Systems and Methods for Algae Concentration,” and also related to U.S. Non-Provisional patent application Ser. No. 13/829,098 filed on Mar. 14, 2013 and entitled “Systems and Methods for Hydrothermal Conversion of Biomass,” and also to U.S. Pat. No. 7,980,024 issued Jul. 19, 2011 and entitled “Photobioreactor Systems Positioned on Bodies of Water” each of the above patent and patent applications are incorporated herein by reference.

### BACKGROUND OF THE INVENTION

1. Field of the Invention

The invention relates generally to the field of renewable energy and more particularly to systems and methods for the production of fuels from algae.

2. Description of the Prior Art

Algae are excellent choices as biomass for the production of biofuels because, among other properties, algae can grow very fast and in areas not well suited for other uses like on bodies of water and on non-arable land. Algal biomass is grown in water, and therefore requires a significant amount of energy to fully dry. Hydrothermal liquefaction (HTL) is a convenient thermo-chemical pathway for the production of algae-derived bio-oils because hydrothermal liquefaction does not require the biomass to be fully dried. However, due to the high protein content of algae, the resulting bio-oils generated by hydrothermal liquefaction are rich in nitrogen, often incorporated into aromatic compounds. Such bio-oils are unsuitable for refining by catalytic deoxygenation processes, those typically employed to produce transportation fuels from petroleum-derived oils, because nitrogen poisons the catalysts. The production of algal biofuels by hydrothermal liquefaction also is lacking in carbon efficiency in that some valuable carbon-containing molecules cannot be efficiently removed from the aqueous phase, and are lost.

Savage et al. (USPGP 2012/0055077) discloses a two-step hydrothermal liquefaction process for converting algae to bio-oil. In the process of Savage et al. a first hydrothermal treatment is performed under subcritical conditions while the second hydrothermal treatment is performed under supercritical conditions.

Gupta et al. (USPGP 2011/0179703) discloses a process for subcritical hydrothermal treatment of biomass to form biochar. Bio-oil is a by-product of the process, and since the bio-oil is not considered desirable, but merely wasted carbon, the bio-oil is recycled back into the subcritical hydrothermal treatment, serving to boost the biochar yield. While the process of Gupta et al. can be used with algae as the biomass, Gupta et al. does not describe using algae and instead lists examples of biomass that yield more significant amounts of biochar such as “forestry or agricultural waste products, wood logs, wood slabs, wood chips, bark, corn-based products, wheat straw, nutshells, [and] sugar cane.” These sources are generally high in lignin, unlike algae.

### SUMMARY

An exemplary system of the present invention comprises a cultivation system configured to produce biomass, such as algae, a treatment system configured to produce an organic phase from the biomass, and a recovery system configured to receive waste from the treatment system, to recover carbon from the waste in the form of carbon dioxide, and to provide the carbon dioxide to the cultivation system. Various embodiments also comprise a refining system configured to receive the organic phase from the treatment system and to produce a fuel therefrom. In some embodiments the cultivation system includes a floating bioreactor, and in other embodiments the system formed by the cultivation system, treatment system, and recovery system is configured to be floated. The system optionally further comprises a dewatering system configured to dewater the biomass produced by the cultivation system. In various embodiments the system can further comprise a carbon dioxide generation system configured to concentrate carbon dioxide out of an input gas stream and to provide the carbon dioxide to the cultivation system. The system can optionally also comprise a hydrogen production system configured to produce molecular hydrogen by electrolysis of water. In embodiments including a hydrogen production system, the treatment system can be further configured to receive the molecular hydrogen.

The recovery system, in various embodiments, is further configured to recover nutrients from the waste and to provide the nutrients to the cultivation system, and/or to direct gaseous waste from the treatment system to the carbon dioxide generation system, and/or includes a digester configured to receive the waste from the treatment system and to produce biogas therefrom. In some of these latter embodiments, the system can further comprise a cogeneration system configured to provide heat and electricity to the treatment system, the cogeneration system further configured to receive at least some of the biogas from the digester. Also in embodiments where the recovery system includes a digester, the system can further comprise a hydrogen production system configured to receive at least some of the biogas from the digester and to produce molecular hydrogen from the methane fraction of the biogas, and in some of these embodiments the hydrogen production system includes a steam methane reformer or a steam gasifier.

The system, in some embodiments, further comprises a carbon dioxide generation system, a hydrogen production system, and a synthesis system. The carbon dioxide generation system is configured to concentrate carbon dioxide out of an input gas stream, the hydrogen production system is configured to produce molecular hydrogen, and the synthesis system is configured to receive at least some of the carbon dioxide from the carbon dioxide generation system and at least some of the molecular hydrogen from the hydrogen production system and to synthesize an olefin from the carbon dioxide and molecular hydrogen.

In those embodiments that include a refining system, the system can additionally comprise a carbon dioxide generation system and a hydrogen production system. The carbon dioxide generation system is configured to concentrate carbon dioxide out of an input gas stream received from the recovery system and to provide the concentrated carbon dioxide to the cultivation system, and the hydrogen production system is configured to provide molecular hydrogen to the refining system. Systems, in some of these embodiments, are further configured to provide waste gases from the refining system to the hydrogen production system.

The system, in still further embodiments, additionally comprises a first hydrothermal reactor configured to receive the biomass and to produce a first mixture including an organic phase and an aqueous phase, and a first separation system configured to separate the organic phase from the aqueous phase and to provide the aqueous phase to the recovery system. In some of these embodiments the first mixture includes a solid phase and the treatment system further includes a second hydrothermal reactor and a second separation system. Here, the second hydrothermal reactor is configured to receive the solid phase and to produce a second mixture including an organic phase and an aqueous phase, and the second separation system is configured to separate the second mixture into the organic phase and the aqueous phase.

The present invention also provides methods for raising biomass and either partially or completely producing fuels therefrom. An exemplary method comprises cultivating a biomass, converting the biomass to a multi-phasic mixture using hydrothermal liquefaction, the multi-phasic mixture including an organic phase and an aqueous phase including dissolved organic compounds, and recovering carbon, in the form of carbon dioxide, from the dissolved organic compounds in the aqueous phase, where cultivating the biomass uses at least some of the carbon dioxide. Cultivating the biomass, in various embodiments, can include concentrating carbon dioxide from the air, from flue gases, and/or from gases produced by the hydrothermal liquefaction in order to provide to the biomass. Cultivating the biomass optionally can include cultivating the biomass with wastewater or with a digestate produced during the recovery of carbon dioxide from the aqueous phase. The method can also comprise comprising recovering nutrients from the aqueous phase, and in these embodiments cultivating the biomass uses at least some of the nutrients.

In some embodiments of the method recovering the carbon includes digesting the dissolved organic compounds to produce biogas. In some of these embodiments recovering the carbon further includes steam methane reforming the biogas or steam gasifying the biogas.

### BRIEF DESCRIPTION OF DRAWINGS

FIG. 1 is a schematic representation of an integrated system of a biorefinery according to an exemplary embodiment of the present invention.

FIG. 2 is a schematic representation of an integrated system of a biorefinery according to another exemplary embodiment of the present invention.

FIG. 3 is a schematic representation of a treatment system and associated recovery system of a biorefinery according to an exemplary embodiment of the present invention.

FIG. 4 is a schematic representation of a treatment system of a biorefinery according to another exemplary embodiment of the present invention.

FIG. 5 is a schematic representation of an integrated biorefinery according to an exemplary embodiment of the present invention.

### DETAILED DESCRIPTION OF THE INVENTION

The present invention provides an integrated biorefinery for the production of fuels from biomass. An exemplary biorefinery of the invention integrates biomass cultivation with processing to convert cultivated biomass into a fuel and integrates further with carbon recovery from the biomass processing. The processing of the biomass into fuels begins in a treatment system, such as a hydrothermal treatment system, that produces an organic phase that is suitable for refining to a fuel and also produces a waste stream. The cultivation system can produce algae as the biomass, for example, and in such embodiments the biorefinery can include a dewatering system to remove sufficient water from the biomass to be acceptable to the treatment system. The biorefinery optionally can comprise a refining system to convert the organic phase to the fuel, and the biorefinery optionally can further comprise a cogeneration system configured to use at least some of the fuel produced by the treatment system to generate electricity and heat that can be used for biorefinery operations.

In various embodiments the biorefinery is configured to efficiently recover components from the waste stream from the treatment system, such as carbon that did not end up in the organic phase, as well as nutrients like nitrogen and phosphorous. The biorefinery recovery systems can additionally collect waste gases from any other system of the biorefiniery, such as the treatment system, and recover chemical species therefrom, in some instances using the same processing as is used to recover such species from the treatment system waste stream. In still further embodiments molecular hydrogen is a product of the recovery system and is reused in the biorefinery, such as in the treatment and refining systems. Molecular hydrogen for the biorefinery can also be produced outside of the recovery system, such as through the electrolysis of water.

In still additional embodiments, the biorefinery comprises a carbon dioxide generation system to provide carbon dioxide to the cultivation system at a concentration above the approximately 395 ppm concentration of atmospheric carbon dioxide for more rapid cultivation. In some of these embodiments the carbon dioxide generation system concentrates carbon dioxide from the atmosphere, from recovered gases from various biorefinery systems, or both. Moreover, various embodiments of the biorefinery system are configured to be floated such on an industrial pond, lake, or sea and to operate in a self-sustaining manner. As such, the ability to efficiently recover and recycle carbon and other components is important to minimize impact on the environment, to reduce reliance on external sources, and to maximize the amount of fuel that is produced.

FIG. 1 schematically illustrates core systems of an exemplary biorefinery 100 of the present invention. The biorefinery 100 comprises a biomass cultivation system 110, a treatment system 120, and a recovery system 130. The biomass cultivation system 110 consumes carbon dioxide and receives sunlight, nutrients, and water to produce a biomass which is then converted into an organic phase by the treatment system 120. The resulting organic phase is suitable for refining, which in some embodiments is performed by the biorefinery 100 while in other embodiments the organic phase is shipped from the biorefinery 100 to be refined elsewhere. The recovery system 130 collects waste products from the treatment system 120 and extracts carbon and optionally other components, such as water and nutrients, and recycles at least some of these back to the cultivation system 110. For example, carbon, which can come out of the treatment system 120 as soluble carbon-compounds in an aqueous solution and/or as carbon-containing gases, can be returned as carbon dioxide to the cultivation system 110.

The biomass cultivation system 110 can comprise, for example, a system for culturing algae, though other forms of biomass can also be cultivated. In various embodiments biomass cultivation system 110 produces algae, such as microalgae, macro algae, blue-green algae, heterotrophic algae, mixotropic algae, cyanobacteria and so forth. Microalgae can be a particularly low-nitrogen form of biomass. In addition to low nitrogen concentrations, biomass characterized by low protein concentration and/or a high lipid concentration are favored for higher bio-oil yield. Algae is generally characterized by a protein concentration of between about 6% to about 50% or more. Other biomass with lower protein concentrations can also be used. Likewise, lipid concentration in algae can generally be between about 5% to about 45%; typically lower for other biomass types.

The biomass cultivation system 110 optionally is open, such as a cultivation pond, or closed, such as a bioreactor. In the case of an open system, only solid or liquid products of the recovery system 130 like a nutrient solution are returned to the biomass cultivation system 110, while recovered carbon dioxide gas can additionally be provided to a closed system. In some embodiments, the biomass cultivation system 110 employs wastewater as a liquid medium in which to grow the biomass, or to provide water and nutrients to biomass cultivated in soil. In these embodiments the biomass cultivation system 110 can also produce cleaned water from the wastewater. Various embodiments of biorefinery 100 are designed to float on water, as described in greater detail below, and in these embodiments the biomass cultivation systems 110 are closed.

Exemplary treatment systems 120 are discussed below in greater detail with respect to FIGS. 3 and 4; exemplary recovery systems 130 are also discussed below in greater detail with respect to FIG. 3. Treatment system 120, in some embodiments, is configured to perform a hydrothermal liquefaction on the received biomass and to further perform a separation on the product thereof. In some instances, a solvent is added to the resulting multi-phasic mixture during the separation following the hydrothermal liquefaction to help extract the organic phase. The result of the separation is the desired organic phase, sometimes mixed with an added solvent, and waste products such as an aqueous phase bearing soluble organic and inorganic compounds and a gas phase comprising carbon dioxide.

An exemplary recovery system 130 is configured to extract as much as can be efficiently recovered from the waste products of the treatment system 120. For instance, the aqueous phase from the treatment system 120 can be provided to a digester to generate biogas, and then the biogas can then be subjected to steam methane reforming to convert the methane (CH₄) fraction to molecular hydrogen and carbon dioxide, or the biogas can be subjected to steam gasification to convert the methane fraction to syngas. Hydroprocessing, direct hydrotreatment, and aqueous chemical processing, such as precipitation from solution, can also be performed by the recovery system 130.

FIG. 2 schematically illustrates another exemplary biorefinery 200 of the present invention. The biorefinery 200 comprises a biomass cultivation system 110, treatment system 120, and recovery system 130 as described above with respect to biorefinery 100. In these embodiments the cultivation system 110 is configured to produce biomass that is generally bound up with more water than is acceptable to the treatment system 120, such as algae when freshly harvested, and therefore the biorefinery 200 additionally comprises a dewatering system 210 configured to remove at least some of the water to produce an aqueous suspension of the biomass. Dewatering of biomass, such as algae, can be performed by such techniques as centrifugation, filtration, and evaporation. Provisional application “Dewatering Systems and Methods for Algae Concentration,” noted above, provides still further systems and processes for algae dewatering. Suitable ranges for the concentration of algae in the aqueous suspension include between about 5% to about 30% of the algae by weight, between about 10% to about 30% of the algae by weight, and between about 25% to about 30% of the algae by weight. Other forms of biomass may also have to be dewatered, or have water added, to create an aqueous biomass suspension within these ranges.

The biorefinery 200 optionally further comprises a carbon dioxide generation system 220 configured to concentrate carbon dioxide out of an input gas stream, either air drawn into the carbon dioxide generation system 220 by a fan or a gas stream provided from a carbon dioxide source. Examples of carbon dioxide sources include oil, coal, and natural gas-fired power plants as well as industrial manufacturing. In some embodiments, carbon dioxide from the recovery system 130 is also admitted to the carbon dioxide generation system 220. In further embodiments, the atmosphere from within the biomass cultivation system 110 is recycled through the carbon dioxide generation system 220. Exemplary carbon dioxide generation systems 220 are described in U.S. Pre-Grant Publication 2012-0174793 published on Jul. 12, 2012 which is incorporated herein by reference.

FIG. 3 schematically illustrates an exemplary treatment system 300 and recovery system 310. The treatment system 300 and recovery system 310 are examples of a suitable treatment system 120 and of a suitable recovery system 130 for biorefineries 100, 200. The treatment system 300 comprises a hydrothermal treatment system 320 and a separation system 330. In some embodiments, both the hydrothermal liquefaction and separation steps are performed in the same vessel, in which case one system serves both functions, while in other embodiments the hydrothermal liquefaction is performed in one vessel and the separation in another.

The hydrothermal treatment system 320 is configured to subject the biomass to a hydrothermal liquefaction process to produce a multi-phasic mixture including an organic phase and an aqueous phase. In various embodiments the hydrothermal liquefaction process takes place at a temperature of between about 150° C. to about 300° C. Optionally, additives such as a catalyst or a pH buffer like K₂CO₃ can be added to the hydrothermal liquefaction process.

The organic phase produced by the hydrothermal liquefaction of biomass is commonly referred to as bio-oil. Hydrothermal liquefaction causes various processes to take place in the biomass, such as cell lysis, hydrolysis of biomolecules, reactions between molecules liberated during hydrolysis, and ultimately the formation of bio-oil by polymerization reactions. Generally, lipids and short organic polymers in the biomass become the bio-oil while the proteins and carbohydrates are water-soluble and are dissolved into the aqueous phase. One benefit of hydrothermal liquefaction is that the biomass does not have to be excessively dried, reducing energy consumption. Additionally, heteroatoms such as nitrogen, sulfur, and phosphorous are preferentially segregated to the aqueous phase by hydrothermal liquefaction.

Examples of separation technologies that are suitable for use in separation system 330 to separate the bio-oil from the aqueous phase include separation tanks, decanters, centrifuges, and filtration. Either or both of the hydrothermal treatment system 320 and the separation system 330 can produce waste gases including carbon dioxide while in operation. Such gases can be directed to the carbon dioxide generation system 220 to recycle that carbon back into the biomass cultivation system 110. In some instances these gases can be fed directly into the biomass cultivation system 110.

The recovery system 310 comprises a cogeneration system 340, a digester 350, a hydrogen production system 360, and the various conduits and manifolds necessary to collect waste gases from the various biorefinery systems such as the hydrothermal treatment system 320 and the separation system 330. The cogeneration system 340 produces heat and electrical power by burning biogas from the digester 350 and to the extent necessary, by burning additional fuel from an external source, such as natural gas. The heat and electrical power can then be provided to the carbon dioxide generation system 220 and hydrothermal treatment system 320, for example. Electrical power from the cogeneration system 340 can also power biomass cultivation system 110, dewatering system 210, and separation system 330. As with other systems, the exhaust from the cogeneration system 340 can be collected and directed to the carbon dioxide generation system 220.

The digester 350 receives the aqueous phase produced by the hydrothermal treatment system 320 and subjects it optionally to aerobic or anaerobic digestion conditions to produce biogas from the soluble carbon compounds carried by the aqueous phase. In addition to being configured to supply at least some of the biogas to the cogeneration system 340, the recovery system 310 can also be configured to supply at least some of the biogas to hydrogen production system 360. The aqueous phase received by the digester 350 also contains dissolved compounds that include heteroatoms such as nitrogen and phosphorous. Accordingly, in addition to the gaseous biogas product, the digester 350 can produce a liquid solution that is enriched with such compounds. This digestate can optionally be recycled to the biomass cultivation system 110.

The recovery system 310 optionally includes a nutrient recovery system 370 comprising, for example, aqueous chemical processing such as precipitation from solution to generate nutrients that can be returned to the biomass cultivation system 110. Nutrient recovery system 370 optionally can upgrade the nutrient value of the recovered nutrients. An alternative to using wastewater in the biomass cultivation system 110 is to direct the wastewater to the digester 350 to mix with the aqueous solution from the treatment system 300; as above, the digestate from the digester 350 can then be directed to the biomass cultivation system 110 to cultivate the biomass. Recycling of water through the systems of an integrated biorefinery beneficially reduces the requirements for additional outside sources of water, reduces environmental impact, and improves system efficiency.

The hydrogen production system 360 can be configured to receive biogas from the digester 350 and in various embodiments either employs steam methane reforming to convert the methane fraction of the biogas to hydrogen and carbon dioxide, or employs steam gasification to convert the methane fraction of the biogas to syngas, a mixture primarily of molecular hydrogen and carbon monoxide. In either case, hydrogen production system 360 optionally can include its own further separation system for separating the molecular hydrogen from the carbon oxide. Molecular hydrogen can be recovered from either gas mixture, such as through pressure swing absorption, and the remaining carbon oxide can be co-fed back into the treatment system 120, or used for biomass cultivation system 110 in the case where the carbon oxide is carbon dioxide. Some or all of the syngas produced by the hydrogen production system 360 is optionally further processed to produce a liquid hydrocarbon. In those embodiments in which molecular hydrogen is separated from carbon dioxide following steam methane reforming, the carbon dioxide can be collected and directed to the carbon dioxide generation system 220.

Syngas produced by the hydrogen production system 360 can optionally be used in a Fischer-Tropsch or synthetic fuels production process to produce liquid hydrocarbons. In other embodiments the syngas can be fed into cogeneration system 340 in addition to, or in the alternative to, the biogas from the digester 350. Solids recovered from separation system 330 optionally also can be subjected to steam gasification separately, or together with the biogas, or together with the aqueous phase, to generate syngas.

Hydrogen production system 360 can optionally obtain molecular hydrogen from water, rather than from the methane in biogas. In these embodiments, hydrogen production system 360 comprises an electrolysis system and rather than receiving biogas from the digester 350, the hydrogen production system 360 can receive electricity from the cogeneration system 340 to split water into molecular hydrogen and molecular oxygen. Also, steam methane reforming or steam gasification can be used to produce hydrogen from externally supplied methane rather than from biogas from the digester 350.

FIG. 4 schematically illustrates an exemplary treatment system 400 as a further example of a suitable treatment system 120 for biorefineries 100, 200. The treatment system 400 comprises a hydrothermal treatment system 320 and a separation system 330, as described above, and additionally another hydrothermal treatment system 410 and a separation system 420. Like hydrothermal treatment system 320 and separation system 330, hydrothermal treatment system 410 and separation system 420 optionally can be separate vessels in fluid communication in continuous or batch operation or the same vessel operated in a batch mode. All four processes can even be performed sequentially in a single vessel, in some embodiments. In treatment system 400, solids separated from the multi-phasic mixture produced by the separation system 330 are directed to the secondary hydrothermal treatment system 410. In various embodiments these solids are primarily comprised of carbohydrates.

The hydrothermal treatment system 410 is configured to subject the solids to a second hydrothermal liquefaction process to produce another multi-phasic mixture again including bio-oil and an aqueous phase. As illustrated for hydrothermal treatment system 320, the biorefinery can be configured to provide heat and electricity from the cogeneration system 340 to the hydrothermal treatment system 410. The second hydrothermal liquefaction process can comprise the same or different processing conditions as in the first hydrothermal liquefaction process. In some embodiments the temperature of the second hydrothermal liquefaction process is greater than the temperature of the first hydrothermal liquefaction process. In various embodiments the second hydrothermal liquefaction process takes place at a temperature of between about 250° C. to about 300° C. Optionally, additives such as a catalyst or a pH buffer like K₂CO₃ can be added.

Separation system 420 separates the bio-oil from the aqueous phase and can include separation tanks, decanters, centrifuges, and filtration, for example. The bio-oil can be directed to refining, while the aqueous phase is directed to the recovery system 130. Gases including carbon dioxide given off by hydrothermal treatment system 410 and separation system 420 can be recovered as described with respect to FIG. 3.

FIG. 5 schematically illustrates another exemplary biorefinery 500 of the present invention including an optional refining system 510 and an optional synthesis system 520. The biorefinery 500 optionally can accept an additional source of biomass 530. The biorefinery 500 comprises a biomass cultivation system 110, treatment system 120, and recovery system 130 as described above with respect to biorefinery 100. The biorefinery 500 further comprises a carbon dioxide generation system 220 as described above with respect to biorefinery 200. It will be appreciated that here, as in previous embodiments, the carbon dioxide generation system 220 can be part of the recovery system 130, outside of the recovery system 130, or both. In those embodiments where the carbon dioxide generation system 220 only concentrates carbon dioxide out of the air or feed gases from external sources, the carbon dioxide generation system 220 is separate from the recovery system 130, and where the carbon dioxide generation system 220 only concentrates carbon dioxide out of gases recovered from sub-systems of the biorefinery 500 the recovery system 130 includes the carbon dioxide generation system 220; as noted previously, the carbon dioxide generation system 220 can also receive both inputs together.

The refining system 510 is configured to receive the organic phase, such as bio-oil, from the treatment system 120, and to receive molecular hydrogen such as from hydrogen production system 360, and to refine the organic phase to a hydrocarbon product such as dodecane, naphtha-like products, gasoline fractions, diesel-like products, and kerosene-like products. The integrated biorefinery 500 can be configured, in some embodiments, to supply heat and electricity to the refining system 510 from the cogeneration system 340. Flue gas from the refining system 510 can include methane and other lightweight gases that can be recovered and passed to either or both of the cogeneration system 340 and/or the hydrogen production system 360. In various embodiments the refining system 510 can employ catalytic hydrogenation/deoxygenation processes (also referred to as hydrotreatments) that are commonly used to refine oils produced from petroleum. In some embodiments, some of the fuel produced by the refining system 510 is used to fire the cogeneration system 340.

Biorefinery 500 also optionally comprises a synthesis system 520. The synthesis system 520 can receive molecular hydrogen and carbon dioxide in order to synthesize synthetic fuels, olefins, methanol, alcohols, and specialty chemicals. The integrated biorefinery 500 can be configured, in some embodiments, to supply heat and electricity to the synthesis system 520 from the cogeneration system 340.

Biorefinery 500 also optionally can consume biomass 530 other than the biomass cultivated by biomass cultivation system 110. This secondary source of biomass 530 can also comprise algae, or can comprise other forms of biomass including biochar, cellulosic biomasses, or waste materials from wastewater treatment such as activated sludge. Biomass 530 can be fed into the treatment system 120 in place of the cultivated biomass, or co-fed into the treatment system 120 along with the cultivated biomass. In some embodiments, oils excreted by the algae cells of certain algae strains comprise the biomass 530.

In various embodiments, the integrated biorefinery floats on a body of water such as an industrial pond, lake, or sea. The integrated biorefinery is configured to float, in some embodiments, by including external floatation devices. Floating bioreactors are described in greater detail in “Photobioreactor Systems Positioned on Bodies of Water.” In various embodiments the entire biorefinery is disposed on a raft, while in other embodiments sub-systems of the biorefinery float separately but are in fluid communication through conduits. In some of these embodiments, the biomass cultivation system 110 comprises a floating bioreactor in fluid communication with a dewatering system 210 or a treatment system 120 disposed on a nearby floating raft. In still further embodiments, some sub-systems like the biomass cultivation system 110 float, while other sub-systems are positioned nearby, either on a shoreline or on a platform over the water, such as a pier or a free-standing structure anchored beneath the water.

In the foregoing specification, the invention is described with reference to specific embodiments thereof, but those skilled in the art will recognize that the invention is not limited thereto. Various features and aspects of the above-described invention may be used individually or jointly. Further, the invention can be utilized in any number of environments and applications beyond those described herein without departing from the broader spirit and scope of the specification. The specification and drawings are, accordingly, to be regarded as illustrative rather than restrictive. It will be recognized that the terms “comprising,” “including,” and “having,” as used herein, are specifically intended to be read as open-ended terms of art.

1. A system comprising:
a cultivation system configured to produce biomass;
a treatment system configured to produce an organic phase from the biomass; and
a recovery system configured to receive waste from the treatment system, to recover carbon from the waste in the form of carbon dioxide, and to provide the carbon dioxide to the cultivation system.

2. The system of claim 1 wherein the cultivation system includes a floating bioreactor.

3. The system of claim 1 wherein the system comprising the cultivation system, treatment system, and recovery system is configured to be floated.

4. The system of claim 1 wherein the recovery system is further configured to recover nutrients from the waste and to provide the nutrients to the cultivation system.

5. The system of claim 1 further comprising a dewatering system configured to dewater the biomass produced by the cultivation system.

6. The system of claim 1 further comprising a carbon dioxide generation system configured to concentrate carbon dioxide out of an input gas stream and to provide the carbon dioxide to the cultivation system.

7. The system of claim 6 wherein the recovery system is further configured to direct gaseous waste from the treatment system to the carbon dioxide generation system.

8. The system of claim 1 wherein the recovery system includes a digester configured to receive the waste from the treatment system and to produce biogas therefrom.

9. The system of claim 8 further comprising a cogeneration system configured to provide heat and electricity to the treatment system, the cogeneration system further configured to receive at least some of the biogas from the digester.

10. The system of claim 8 further comprising a hydrogen production system configured to receive at least some of the biogas from the digester and to produce molecular hydrogen from the methane fraction of the biogas.

11. The system of claim 10 wherein the hydrogen production system includes a steam methane reformer.

12. The system of claim 10 wherein the hydrogen production system includes a steam gasifier.

13. The system of claim 1 further comprising a hydrogen production system configured to produce molecular hydrogen by electrolysis of water.

14. The system of claim 1 further comprising
a carbon dioxide generation system configured to concentrate carbon dioxide out of an input gas stream,
a hydrogen production system configured to produce molecular hydrogen, and
a synthesis system configured to receive at least some of the carbon dioxide from the carbon dioxide generation system and at least some of the molecular hydrogen from the hydrogen production system and to synthesize an olefin from the carbon dioxide and molecular hydrogen.

15. The system of claim 1 further comprising a hydrogen production system configured to produce molecular hydrogen, the treatment system being further configured to receive the molecular hydrogen.

16. The system of claim 1 further comprising a refining system configured to receive the organic phase from the treatment system and to produce a fuel therefrom.

17. The system of claim 16 further comprising
a carbon dioxide generation system configured to concentrate carbon dioxide out of an input gas stream received from the recovery system and to provide the concentrated carbon dioxide to the cultivation system, and
a hydrogen production system configured to provide molecular hydrogen to the refining system.

18. The system of claim 17 further configured to provide waste gases from the refining system to the hydrogen production system.

19. The system of claim 1 wherein the treatment system includes
a first hydrothermal reactor configured to receive the biomass and to produce a first mixture including an organic phase and an aqueous phase; and
a first separation system configured to separate the organic phase from the aqueous phase and to provide the aqueous phase to the recovery system.

20. The system of claim 19 wherein the first mixture includes a solid phase and the treatment system further includes
a second hydrothermal reactor configured to receive the solid phase and to produce a second mixture including an organic phase and an aqueous phase, and
a second separation system configured to separate the second mixture into the organic phase and the aqueous phase.

21-30. (canceled)
